(19) United States
(12) Patent Application Publication
(10) Pub. No.: US 2019/0196411 A1
(43) Pub. Date: Jun. 27, 2019

(54) SMARTWATCH ASSEMBLIES HAVING ELECTROCARDIOGRAM SENSORS, PHOTOPLETHYSMOGRAPHY SENSORS, AND BLOOD PRESSURE MONITORS AND RELATED METHODS

(71) Applicant: Michael M. Yuen, Hong Kong (CN)

(72) Inventor: Michael M. Yuen, Hong Kong (CN)

(21) Appl. No.: 15/855,423

(22) Filed: Dec. 27, 2017

(51) Int. Cl. G04B 47/06; A61B 5/0408; A61B 5/024; A61B 5/00

(52) U.S. Cl. CPC G04B 47/063; A61B 5/681; A61B 5/02416; A61B 5/04085

(57) ABSTRACT

A smartwatch assembly including an outer frame portion and an insert portion removably insertable into the outer frame portion. The insert portion may include a casing, a controller disposed within the casing, an electrocardiogram sensor operably coupled to the controller, the electrocardiogram sensor having at least two electrodes configured to be placed in contact with a user's skin, a photoplethysmography sensor operably coupled to the controller and oriented to face the user's skin, and a display operably coupled to the controller and configured to show data related to measurements taken by the electrocardiogram sensor and the photoplethysmography sensor. The photoplethysmography sensor may detect trigger events in a heart function of the user, and, in response to the detection of a trigger event, the electrocardiogram sensor may initiate an electrocardiogram measurement of the user.

### TECHNICAL FIELD

This disclosure relates generally to smartwatch assemblies and methods of making smartwatch assemblies. Specifically, this disclosure relates to smartwatch assemblies that have sensors for monitoring a user's heart functions and providing measurements of heart function.

### BACKGROUND

Smartwatches are wristwatches that have functionality beyond timekeeping. Some smartwatches are portable media players, and some smartwatches run mobile apps using a mobile operating system. Smartwatches often include electronic displays where a user can interface with the smartwatches and control their functionality. Smartwatches also often include sensors to track a user's activity and inactivity.

### BRIEF SUMMARY

Some embodiments of the present disclosure include a smartwatch assembly. The smartwatch assembly may include an outer frame portion and an insert portion removably insertable into the outer frame portion. The insert portion may include a casing, a controller disposed within the casing, an electrocardiogram sensor operably coupled to the controller, the electrocardiogram sensor having at least two electrodes configured to be placed in contact with a user's skin, a photoplethysmography sensor operably coupled to the controller and oriented to face the user's skin, and a display operably coupled to the controller and configured to show data related to measurements taken by the electrocardiogram sensor and the photoplethysmography sensor. The photoplethysmography sensor may detect trigger events in a heart function of the user, and, in response to the detection of a trigger event, the electrocardiogram sensor may initiate an electrocardiogram measurement of the user.

Some embodiments of the present disclosure include a smartwatch assembly. The smartwatch assembly may include an outer frame portion and an insert portion removably insertable into the outer frame portion. The insert portion may include a casing, a controller disposed within the casing, an electrocardiogram sensor operably coupled to the controller, the electrocardiogram sensor having at least two electrodes configured to be placed in contact with a user's skin, a photoplethysmography sensor operably coupled to the controller and oriented to face the user's skin, and a display operably coupled to the controller and configured to show data related to measurements taken by the electrocardiogram sensor and the photoplethysmography sensor. The photoplethysmography sensor may detect trigger events in a heart function of the user, and, in response to the detection of a trigger event, the smartwatch assembly may alert the user of the detected trigger event via the display. Additionally, in response to a user interaction, the electrocardiogram sensor may initiate an electrocardiogram measurement.

Some embodiments of the present disclosure include a smartwatch assembly system. The smartwatch assembly system may include at least one processor and at least one non-transitory computer readable storage medium storing instructions thereon that, when executed by the at least one processor, cause the system to: monitor a user's heart function via a photoplethysmography sensor; detect a trigger event related to the user's heart function via the photoplethysmography sensor; in response to detecting the trigger event, initiate an electrocardiogram measurement of the user via an electrocardiogram sensor; and show data related to the electrocardiogram measurement via a display.

### BRIEF DESCRIPTION OF THE DRAWINGS

The patent or application file contains at least one drawing executed in color. Copies of this patent or patent application publication with color drawings will be provided by the Office upon request and payment of the necessary fee.

For a detailed understanding of the present disclosure, reference should be made to the following detailed description, taken in conjunction with the accompanying drawings, in which like elements have generally been designated with like numerals, and wherein:

FIG. 1A is a front perspective view of a smartwatch assembly according to an embodiment of the present disclosure;

FIG. 1B is a back perspective view of the smartwatch assembly of FIG. 1A.

FIG. 2 is a schematic representation of a smartwatch assembly control system according to one or more embodiments of the present disclosure;

FIG. 3A is a perspective view of an insert portion of a smartwatch assembly according to one or more embodiments of the present disclosure;

FIG. 3B is another perspective view of the insert portion of FIG. 3A;

FIG. 4 is a back perspective view of the smartwatch assembly of FIG. 1A;

FIG. 5 is a front view of a display of the smartwatch assembly of FIG. 1A;

FIG. 6A is a perspective view of a smartwatch assembly according to an embodiment of the present disclosure; and

FIG. 6B is another perspective view of the smartwatch of FIG. 6A.

### DETAILED DESCRIPTION

The illustrations presented herein are not actual views of any particular smartwatch assembly, or any component thereof, but are merely idealized representations, which are employed to describe the present invention.

As used herein, any relational term, such as “first,” “second,” “adjacent,” “front,” “rear,” etc., is used for clarity and convenience in understanding the disclosure and accompanying drawings, and does not connote or depend on any specific preference or order, except where the context clearly indicates otherwise. For example, these terms may refer to an orientation of elements of the smartwatch assembly when the smartwatch assembly is being worn by a user on the user's arm in a conventional manner for wearing watches.

Some embodiments of the present disclosure include a smartwatch assembly that includes an electrocardiogram sensor (“ECG sensor”) that may be operably coupled to a controller and may include at least two electrodes for contacting a user's skin (e.g., a user wearing the smartwatch assembly). The ECG sensor may include a sensor for detecting and recording electrical activity generated by heart muscle depolarization of a user, which propagate in pulsating electrical waves toward the skin of a user. Moreover, the ECG sensor may be used for obtaining an electrocardiogram measurement from the user, including the parameters pertaining to the “QRS complex” and/or PQRST waveform of the acquired electrocardiogram measurement. In some embodiments, the at least two electrodes of the ECG sensor may extend through (i.e., may be exposed through) a back cover of the smartwatch assembly. In additional embodiments, at least one electrode of the at least two electrodes may extend through (i.e., may be exposed on) a lateral side of the smartwatch assembly. Having at least one electrode of the at least two electrodes be accessible via an opposite hand of the user and another electrode contacting the skin of the wrist of the user wearing the smartwatch assembly may expand locations where electrical activity is detected by the ECG sensor and may enable a more accurate measurement by the ECG sensor.

One or more embodiments of the present disclosure include a smartwatch assembly that further includes a photoplethysmography sensor (“PPG sensor”) that may be operably coupled to the controller. For instance, the PPG sensor may include a sensor for optically detecting changes in a blood flow volume through a tissue via reflection from or transmission through the tissue. The PPG sensor may associate changes in light intensity with small variations in blood perfusion, and as a result, heart beats. In some embodiments, the PPG sensor may extend through a back cover of the smartwatch assembly and may be oriented to face a user's skin. The PPG sensor may monitor heart rate variability of a user. For example, the PPG sensor may monitor HRV continuously. Furthermore, based on the monitored heart rate variability (HRV), the PPG sensor and/or controller may detect and/or determine trigger events (e.g., heart irregularities) indicated in the heart function of the user. In response to a detection of a trigger event via the PPG sensor, the controller may initiate an ECG measurement via the ECG sensor. In additional embodiments, in response to a detection of a trigger event via the PPG sensor, the controller may alert a user of the trigger event via a display of the smartwatch assembly.

Additional embodiments of the present disclosure include a smartwatch assembly having an insert portion including the ECG and PPG sensors that can be switched between (e.g., inserted into and removed from) an outer frame portion connected to a wrist band and an outer frame portion connected to a heart rate strap. In some embodiments, the heart rate strap may include at least one electrode of the ECG sensor of the smartwatch assembly. For example, the heart rate strap may include one or more portions of conductive rubber that comprise the at least one electrode of the ECG sensor. Accordingly, the smartwatch assemblies of the present disclosure may be advantageous over conventional smartwatch assemblies. For example, upon being notified by the smartwatch assembly of a detected heart irregularity, the user may remove the insert portion from the outer frame portion connected to the wristband and may insert the insert portion into the outer frame portion connected to the heart rate strap to acquire a more accurate ECG measurement. Based on the more accurate ECG measurement, a user may decide whether or not to seek medical attention. Accordingly, the smartwatch assemblies of the present disclosure may provide a useful and potentially lifesaving monitor to users who may be prone to heart irregularities and/or heart attacks.

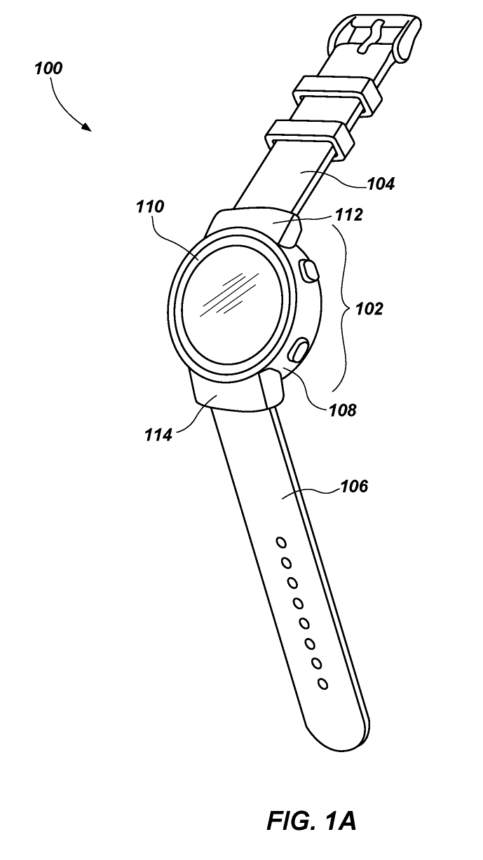

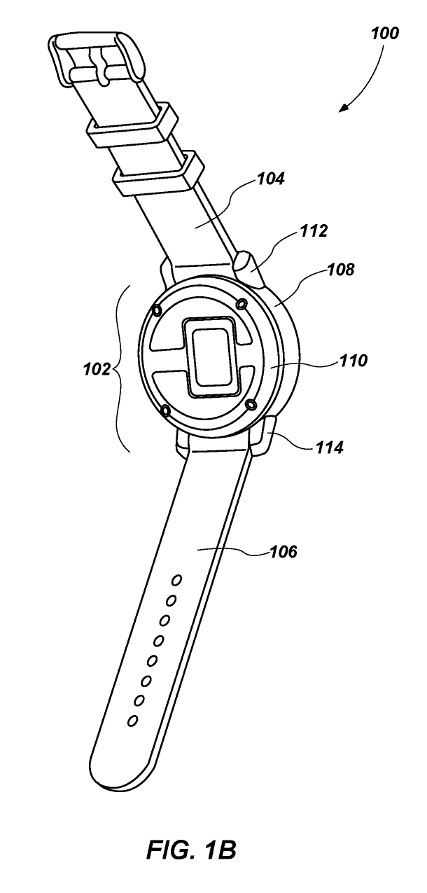

FIG. 1A shows a front perspective view of a smartwatch assembly 100 according to an embodiment of the present disclosure. FIG. 1B shows a back perspective view of the smartwatch assembly 100. Referring FIGS. 1A and 1B together, the smartwatch assembly 100 may include a watch body 102, a first band portion 104, and a second band portion 106.

The watch body 102 may include a generally annular-outer frame portion 108 (e.g., receptacle portion), an insert portion 110, a first lug 112, and a second lug 114. As is described in greater detail below, the insert portion 110 may be removably insertable into the outer frame portion 108. The first and second lugs 112, 114 may extend out radially from the outer frame portion 108 of the watch body 102 on opposite sides of the watch body 102. The first band portion 104 may be coupled to the first lug 112, and the second band portion 106 may be coupled to the second lug 114. The first and second band portions 104, 106 may be sized and shaped to be wrapped around a wrist of a user and to fasten the smartwatch assembly 100 to the wrist of the user.

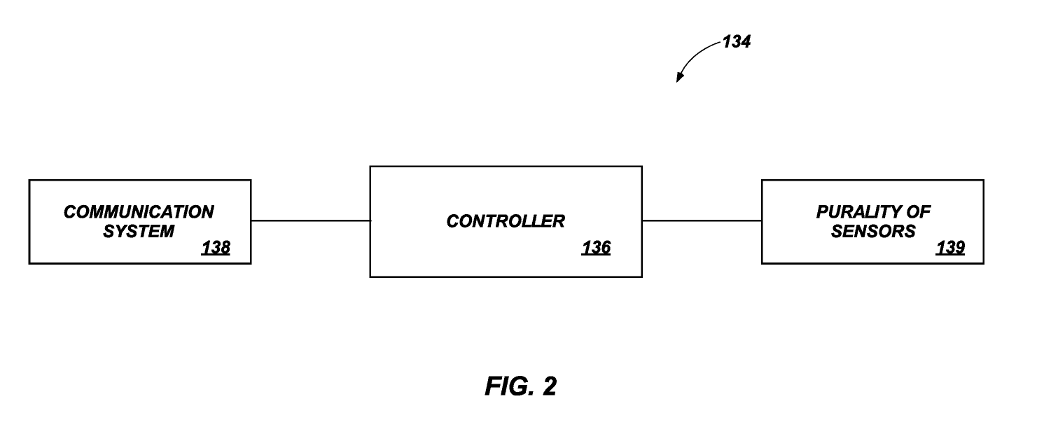

FIG. 2 is a schematic representation of a smartwatch control system 134 that may be at least partially disposed within the insert portion 110 of the smartwatch assembly 100. The smartwatch control system 134 may include a controller 136, a communication system 138, and a plurality of sensors 139.

The controller 136 may include one or more of special-purpose or general-purpose computer including computer hardware, such as, for example, one or more processors and system memory, as discussed in greater detail below. The controller 136 may also include physical and other computer-readable media for carrying or storing computer-executable instructions and/or data structures. In particular, one or more of the processes described herein may be implemented at least in part as instructions embodied in a non-transitory computer-readable medium and executable by the controller 136. In general, a processor (e.g., a microprocessor) receives instructions, from a non-transitory computer-readable medium, (e.g., a memory, etc.), and executes those instructions, thereby performing one or more processes, including one or more of the processes described herein.

Referring to FIGS. 1A, 1B, and 2 together, the communication system 138 of the smartwatch control system 134 may be operably coupled to the controller 136 and may enable the smartwatch assembly 100 to wirelessly communicate with other devices. For example, the communication system 138 may enable the smartwatch control system 134 to communicate with other devices through Wi-Fi, BLUETOOTH® 2.0, BLUETOOTH® low energy (“BLE”) 4.0, infrared communication, ANT, ANT+, etc. In some embodiments, the communication system 138 may enable the smartwatch control system 134 to communicate with a smartphone, such as, for example, an IPHONE® or an ANDROID® phone. For example, the controller 136 may be able to communicate with devices using IOS® software and/or ANDROID® software.

In some embodiments, an application (or “app”) specific to the smartwatch assembly 100 may be included on a smartphone (hereinafter “SW app”) and may allow a user to customize features of the smartwatch assembly 100 from the smartphone. For example, the SW app may include the SW app described in U.S. Pat. No. 9,841,735, to Yuen, issued Dec. 12, 2017, the disclosure of which is incorporated in its entirety by this reference herein.

In some embodiments, the SW app and/or smartwatch control system 134 of the smartwatch assembly 100 may interface with, for example, the HEALTH KIT® App and/or the GOOGLE FIT® App or any other app designed to track a user's activity. As used herein, the term “activity” may refer to physical activity such as walking, running, swimming, burning calories, etc. Furthermore, the term “activity” may include other activities such as sleeping. In some embodiments, the smartwatch control system 134 may communicate with and interface with other apps on a smartphone, such as, for example, mail apps, texting apps, call placing and receiving apps, sleep tracking apps, map apps, alarm apps, and global positioning apps.

Moreover, the smartwatch control system 134 may access data on the smartphone such as, for example, global positioning data, activity data, usage data, etc. In some embodiments, the smartwatch control system 134 may be in at least substantially constant wireless communication with the smartphone. In some embodiments, the smartwatch assembly 100 may be able to stay in constant communication with the smartphone when the smartwatch assembly 100 is within approximately 50 meters of the smartphone. In some embodiments, the smartwatch assembly 100 may be able to stay in constant communication with the smartphone when the smartwatch assembly 100 is within approximately 100 meters of the smartphone. In some embodiments, the smartwatch assembly 100 may be able to stay in constant communication with the smartphone when the smartwatch assembly 100 is within approximately 150 meters of the smartphone.

Although the smartwatch assembly 100 is described herein as communicating with a smartphone, embodiments of the present disclosure may not be so limited. For example, the smartwatch assembly 100 may communicate and may interface with one or more of a computer, a laptop, a personal digital assistant, a pedometer, and other mobile devices such as a FITBIT®, JAWBONE®, and other smartwatches. To facilitate explanation of the smartwatch assembly 100, the smartwatch assembly 100 will be described herein as communicating and interfacing with a smartphone. However, it is understood that that smartwatch assembly 100 may communicate and interface with any of the above-listed devices.

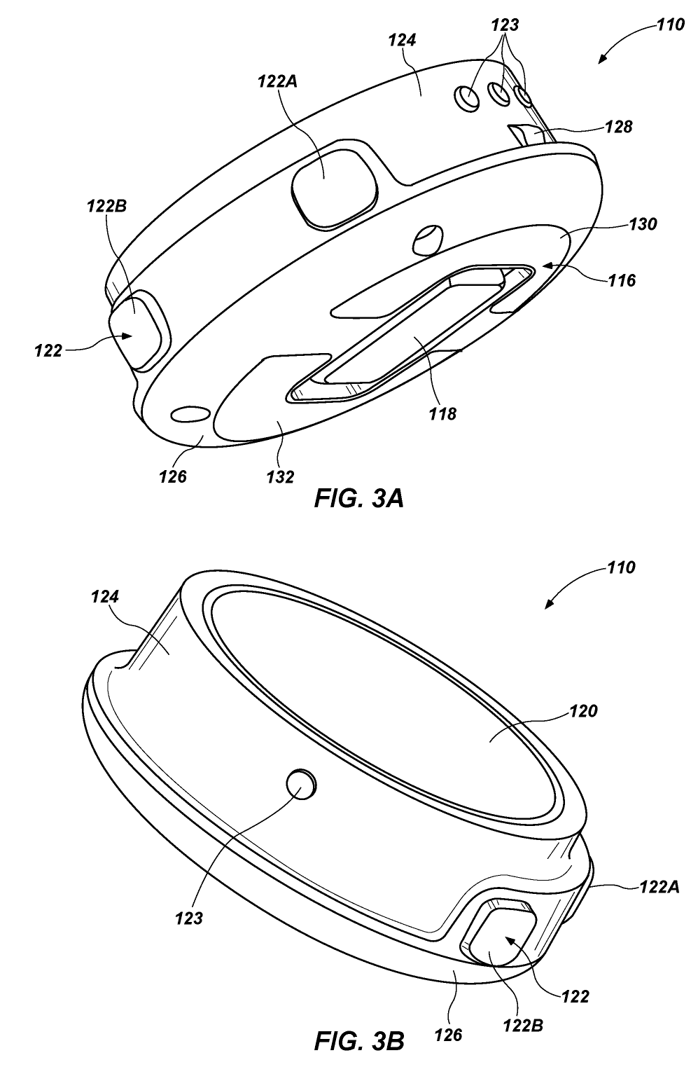

The plurality of sensors 139 may be operably coupled to the controller 136 and may provide data related to a user's activities, inactivity, and health to the controller 136. The plurality of sensors 139 are described in greater detail below in regard to FIGS. 3A and 3B.

FIG. 3A shows a perspective view of the insert portion 110 of a smartwatch assembly 100 (FIG. 1A) according to one or more embodiments of the present disclosure. FIG. 3B shows another perspective view of the insert portion 110 of FIG. 2A. Referring to FIGS. 1A-3B together, the insert portion 110 of the smartwatch assembly 100 (e.g., smartwatch assembly 100) may include a controller 136, an electrocardiogram sensor 116, a photoplethysmography sensor 118, a display 120, a plurality of control mechanisms 122, a generally annular side cover 124, a back cover 126, and a plurality of securing mechanisms 128. In some embodiments, the smartwatch assembly 100 may further include a vibrator. For example, the smartwatch assembly 100 may include the vibrator described in U.S. Pat. No. 9,841,735, to Yuen, issued Dec. 12, 2017, the disclosure of which is incorporated in its entirety by this reference herein. As is discussed in greater detail below, the vibrator may be operably coupled to the controller 136 and may be used (e.g., caused to vibrate) in response to certain events, such as, an alarm of the smartphone and the smartphone receiving a text, email, voicemail, and/or phone call. Additionally, the smartwatch assembly 100 may include an additional sensors (e.g., sensors of the plurality of sensors 139), and the additional sensors may be operably coupled to the controller 136 and may include a magnetic pendulum (i.e., pedometer), motion sensors, etc. For example, the additional sensors may include at least one multi-axis accelerometer. In some embodiments, the accelerometer may include at least 3 axes. In some embodiments, the accelerometer may include at least 6 axes.

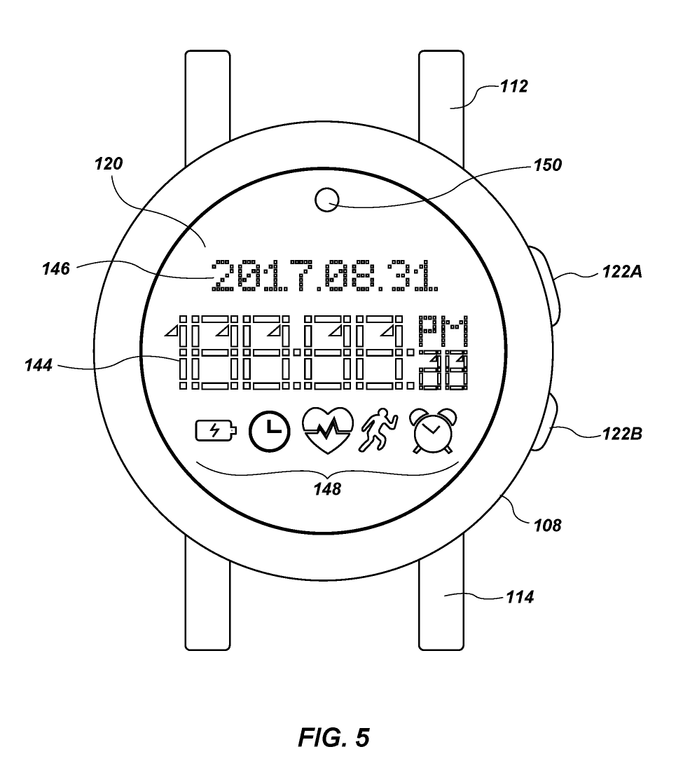

The display 120 may be attached to a first side of the annular side cover 124 and the back cover 126 may be attached to a second opposite side of the annular side cover 124. The display 120 may be operably connected to the controller 136. The display 120 may allow a user to provide input to, receive output (e.g., alerts) from, and otherwise transfer data to and receive data from controller 136. In some embodiments, the display 120 may include a display screen and/or a touch screen. The display 120 may also include one or more devices for presenting output to a user, including, but not limited to, a graphics engine, one or more output drivers (e.g., display drivers), one or more audio speakers, and one or more audio drivers. In certain embodiments, the display 120 is configured to provide graphical data to a display screen for presentation to a user. The graphical data may be representative of one or more graphical user interfaces and/or any other graphical content as may serve a particular implementation. The display 120 may also include one or more LED modules. The display 120 is described in greater detail in regard to FIG. 5.

The electrocardiogram sensor 116 (“ECG sensor 116”) may be operably coupled to the controller 136 (FIG. 2) and may include at least two electrodes 130, 132 for contacting a user's skin (e.g., a user wearing the smartwatch assembly 100). The ECG sensor 116 may include any known ECG sensor 116 known in the art. For instance, the ECG sensor 116 may include a sensor for detecting and recording electrical activity generated by heart muscle depolarization of a user, which propagate in pulsating electrical waves towards the skin of a user. Moreover, as is discussed in further detail below, the ECG sensor 116 may be used for obtaining an electrocardiogram measurement from the user, including the parameters pertaining to the “QRS complex” and/or PQRST waveform of the acquired electrocardiogram measurement. The smartwatch assembly 100 may include algorithms stored in memory, which may be executed by a processor of the controller 136 (FIG. 2) using the acquired electrocardiogram data to provide information to the user (via the display 120) pertaining to one or more of the following variables: a heart rate, an effective heart age (as opposed to actual heart age), a heart rate robustness, an R to R interval, a heart rate variability, a quality of the electrocardiogram signal, and mood, fatigue, and stress levels of the user. Additionally, the algorithms may, when executed by the processor of the controller 136 (FIG. 2), diagnose various heart conditions including atrial fibrillation, heart arrhythmia, etc.

In some embodiments, the at least two electrodes 130, 132 of the ECG sensor 116 may extend through (i.e., may be exposed through) the back cover 126 of the insert portion 110. In some instances, each electrode of the at least two electrodes 130, 132 may have a general half-moon shape. In additional embodiments, at least one electrode of the at least two electrodes 130, 132 may extend through (i.e., may be exposed on) a lateral side of the insert portion 110. For instance, the at least one electrode of the at least two electrodes 130, 132 may extend through the annular side cover 124 of the insert portion 110. As another example, the at least one electrode of the at least two electrodes 130, 132 may comprise a portion of the annular side cover 124. Furthermore, the at least one electrode of the at least two electrodes 130, 132 may be exposed through the outer frame portion 108 of the watch body 102 of the smartwatch assembly 100. For example, the at least one electrode of the at least two electrodes 130, 132 may be a bezel, a control mechanism 122 (e.g., one of the plurality of control mechanisms 122), and/or a portion of the outer frame portion 108 of the smartwatch assembly 100. Accordingly, the at least one electrode of the at least two electrodes 130, 132 may be accessible via an opposite hand of the user (e.g., a hand of an arm of the user not wearing the smartwatch assembly 100). In such embodiments, the ECG sensor 116 may include at least three electrodes. Furthermore, the electrode exposed through the outer frame portion 108 of the watch body 102 of the smartwatch assembly 100 may include a positive terminal of the ECG sensor 116. One of the electrodes extending through the back cover 126 of the insert portion 110 may include a negative terminal of the ECG sensor 116, and the other electrode extending through the back cover 126 may include a ground terminal of the ECG sensor 116. Having the at least one electrode of the at least two electrodes be accessible via an opposite hand of the user and another electrode contacting the skin of the wrist of the user wearing the smartwatch assembly 100 may expand locations where electrical activity is detected by the ECG sensor 116 and may enable a more accurate measurement by the ECG sensor 116.

Via contact with a user's skin, the ECG sensor 116 may perform (i.e., take) an electrocardiogram measurement of a user wearing the smartwatch assembly 100. For instance, the ECG sensor 116 may be configured to detect and measure a heart rate, an interbeat interval, and a heart rate variability of a user. Furthermore, the ECG sensor 116 may be configured to generate a graph of voltage over time of the heart muscle beating (i.e., an “EKG”). The graph may include a P-wave, a QRS complex, and a T-wave, which are known in the art. Additionally, as mentioned above, the ECG sensor 116 and/or controller 136 may determine, based on ECG measurements, an effective heart age, a heart rate robustness, an R to R interval, a heart rate variability, a quality of the electrocardiogram signal, a mood of the user, fatigue level of the user, and stress levels of the user. Likewise, the ECG sensor 116 and/or controller 136 may determine, based on ECG measurements, an estimated inebriation level, a Zen index, a detection of inebriation, a drowsiness level, a sleep quality, heart irregularities, a recovery rate, and an oxygen saturation of the user. Moreover, when in use, the ECG sensor 116 and/or controller 136 may cause data related to an ECG measurement and any additional determined data to be displayed on the display 120 for inspection by a user.

In some embodiments, the controller 136 may utilize the display 120 to provide feedback to a user while the user is attempting to take an ECG measurement. For example, the controller 136 may cause one or more colors to be displayed on the display 120 to indicate different statuses related to the ECG measurement. For instance, the controller 136 may cause the display 120 to display a first (e.g., red) color (e.g., illuminate a first LED module) when the ECG sensor 116 could not acquire and/or read a signal properly. Additionally, the controller 136 may cause the display 120 to display a second (e.g., amber) color when the ECG sensor 116 is actively acquiring and/or reading an ECG signal. Furthermore, the controller 136 may cause the display 120 to display a third (e.g., green) color when the smartwatch assembly 100 is properly placed for acquiring an ECG signal and the ECG measurement has been taken. The vibrator (described above) may also vibrate upon completion of the acquisition of the ECG measurement.

Additionally, the controller 136 may utilize the display 120 to provide feedback (e.g., information) pertaining to the parameters (e.g., heart rate, effective heart age, heart rate robustness, etc.) listed above as determined using the algorithms and ECG measurement. For instance, the controller 136 may cause the display 120 to display a first (e.g., red) color when the ECG measurement and associated parameters appear to indicate an abnormality (e.g., heart irregularity). Additionally, the controller 136 may cause the display 120 to display a second (e.g., amber) color when the ECG measurement and associated parameters potentially indicate an abnormality (e.g., heart irregularity). Furthermore, the controller 136 may cause the display 120 to display a third (e.g., green) color when the ECG measurement and associated parameters appear normal. In some embodiments, the colors may blink. As a non-limiting example, the colors may blink while a measurement is being taken, and the colors may stop blinking and may remain solid (i.e., not blink) to indicate an outcome of the ECG measurement. The display 120 is described in further detail below in regard to FIG. 5. Additionally, as is be described in further detail below, data pertaining to the ECG measurement and associated parameters may be transmitted to the smartphone or other associated device for further viewing, review, and tracking by the user.

In some embodiments, the ECG sensor 116 and controller 136 may also be able to detect that the user is possibly intoxicated (e.g., by alcohol, medications, etc.) via the ECG measurement. For example, algorithms have been disclosed for estimating intoxication using ECG data. See, e.g., C. K. Wu et al., A Precise Drunk Driving Detection Using Weighted Kernel Based On Electrocardiogram, Sensors 2016, 16, 659, the contents of which are incorporated herein in their entirety by reference herein. In such an embodiment, the smartwatch assembly 100 may utilize motion sensors to detect that the user is potentially driving a vehicle, and then perform an ECG to determine whether or not the user is potentially intoxicated. If the smartwatch assembly 100 detects that the user is potentially intoxicated, the smartwatch assembly 100 may alert the user by using the display 120 and/or vibrator.

In some embodiments, the smartwatch assembly 100 may have an operational mode in which the controller 136 may periodically cause the ECG sensor 116 to initiate and take ECG measurements and analyze the acquired ECG measurements for heart irregularities. As a result, users who are known to be prone to heart irregularities or who are at risk for a heart attack, may place the smartwatch assembly 100 into the operational mode to periodically monitor the user's heart function and analyze the acquired signals for heart irregularities. In the event an irregularity is detected, the controller 136 may cause an alert to be displayed via the display 120 and/or the vibrator.

The photoplethysmography sensor 118 (“PPG sensor 118”) may be operably coupled to the controller 136 (FIG. 2). The PPG sensor 118 may include any PPG sensor 118 known in the art. For instance, the PPG sensor 118 may include a sensor for optically detecting changes in a blood flow volume through a tissue via reflection from or transmission through the tissue. The PPG sensor 118 may associate changes in light intensity with small variations in blood perfusion, and as a result, heart beats.

In one or more embodiments, the PPG sensor 118 may extend through the back cover 126 of the insert portion 110 and may be oriented to face a user's skin. In some embodiments, the PPG sensor 118 may extend through the back cover 126 in a location in-between electrodes of the at least two electrodes 130, 132 of the ECG sensor 116. The PPG sensor 118 may monitor heart rate variability of a user. For example, the PPG sensor 118 may monitor HRV continuously. Furthermore, based on the monitored heart rate variability (HRV), the PPG sensor 118 and/or controller 136 may detect and/or determine trigger events indicated in the heart function of the user. For instance, the PPG sensor 118 and/or controller 136 may determine one or more trigger events indicated in the heart function of the user via one or more heart rate algorithms. The trigger events may include one or more of reduced (i.e., low) HRV and/or irregular heart rhythm. In view of the foregoing, the PPG sensor 118 may continuously monitor heart function of the user, and the ECG sensor 116 may be utilized periodically, when a potential irregularity is detected by the PPG sensor 118, and/or when initiated by the user.

In some embodiments, in response to a detection of a trigger event via the PPG sensor 118, the controller 136 may initiate an ECG measurement via the ECG sensor 116. In additional embodiments, in response to a detection of a trigger event via the PPG sensor 118, the controller 136 may alert a user of the trigger event via the display 120. For instance, the controller 136 may cause one or more alert messages or lights to be shown on the display 120. In further embodiments, in response to a detection of a trigger event via the PPG sensor 118, the controller 136 may prompt the user to initiate an ECG measurement. For example, the controller 136 may cause a message prompting the user to initiate an ECG measurement on the display 120.

As mentioned above, the smartwatch assembly 100 may track additional activities of the user. In some embodiments, the smartwatch assembly 100 may acquire data required to track a user's activity from the ECG sensor 116, the PPG sensor 118, and the plurality of sensors 139 (FIG. 2) included in the in the insert portion 110 of the watch body 102 of the smartwatch assembly 100. In some embodiments, the smartwatch assembly 100 may acquire data required to track a user's activity from a smartphone. For example, as noted above, the SW app may interface with other apps (e.g., HEALTH KIT® and GOOGLE FIT®) and functions (e.g., global positioning) of the smartphone to acquire data required to track a user's activity. In other words, in some embodiments, the SW app may track an activity with the smartphone and the smartwatch assembly 100 may indicate the activity tracked by the SW app on the smartphone. In some embodiments, the smartwatch assembly 100 may acquire data required to track a user's activity from both of the plurality of sensors 139 (FIG. 2) and a smartphone. Additionally, as noted above, the smartwatch assembly 100 may transmit data pertaining to a user's activities, ECG measurements, PPG measurements, and inactivity to a smartphone or other associated device for further viewing, review, and tracking by the user.

Additionally, the smartwatch assembly 100 may notify a user of communication received via a smartphone. For example, the controller 136 may cause one or more messages to be displayed on the display 120 and/or the vibrator to vibrate in response to one or more events identified or created by the smartphone (referred to herein as “alerts”) or the smartwatch assembly 100. For example, the controller 136 may cause a message to be displayed on the display 120 when a text, phone call, email, and/or voicemail is received on the smartphone. Furthermore, in some embodiments, controller 136 may cause a message to be displayed on the display 120 in response to activity performed by the user and as measured (e.g., tracked) by the smartphone or smartwatch assembly 100. In other words, the smartwatch assembly 100 may track an activity performed by the user and may indicate tracked (e.g., measured, recorded, sensed, etc.) activity to the user by showing a message on the display 120. For example, the controller 136 may cause a message indicating to a user a quantity and/or quality of an activity (e.g., walking, running, swimming, calories burned, sleeping, etc.) performed by the user to be displayed on the display 120.

Referring still to FIGS. 1A-3B together, the plurality of control mechanisms 122 may extend radially outward from the annular side cover 124. Furthermore, when assembled with the outer frame portion 108, the plurality of control mechanisms 122 may be disposed in and extend through a plurality of holes in the outer frame portion 108 and may be operably coupled to the controller 136. Accordingly, the plurality of control mechanisms 122 may be accessible through the outer frame portion 108 of the watch body 102. In some embodiments, the plurality of control mechanisms 122 may include one or more of a button, a switch, and a crown.

A first control mechanism 122A of the plurality of control mechanisms 122 may be oriented between about a one o'clock position and a three o'clock position. A second control mechanism 122B of the plurality of control mechanisms 122 may be oriented between about a three o'clock position and a five o'clock position. In some embodiments, the plurality of control mechanisms 122 may be operably coupled to the controller 136. Furthermore, via interaction with (e.g., pressing) the plurality of control mechanisms 122, a user may initiate measurements with the PPG or ECG sensors 116, 118 and/or change modes of the smartwatch assembly 100.

The operation of the plurality of control mechanisms 122 is described in greater detail in regard to FIG. 5.

In some embodiments, the plurality of securing mechanisms 128 may extend radially outward from the annular side cover 124 of the insert portion 110 and may be sized and shaped to engage the outer frame portion 108 of the smartwatch assembly 100 and to removably secure the insert portion 110 within the outer frame portion 108 of the smartwatch assembly 100. For instance, the plurality of securing mechanisms 128 may include a plurality of protrusions.

In one or more embodiments, the insert portion 110 may include a plurality of contacts 123 for electrically connecting portions of the ECG sensor 116 and for providing charging pins. For example, one or more of the plurality of contacts 123 may electrically connect one or more electrodes located away from the insert portion 110 to the insert portion 110 and controller 136. As a non-limiting example, one or more of the plurality of contacts 123 may electrically connect an electrode located in a band portion (e.g., the first band portion 104 or second band portion 106) of the smartwatch assembly 100 to the insert portion 110 and controller 136. Additionally, one of more of the plurality of contacts 123 may provide charging pins for contact with a charging dock.

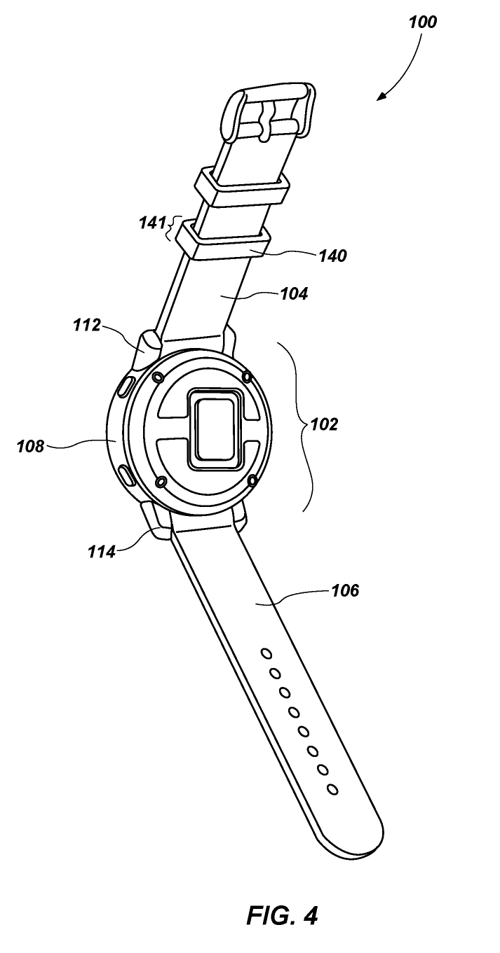

FIG. 4 shows a back perspective view of the smartwatch assembly 100 of FIGS. 1A-3B. In some embodiments, the smartwatch assembly 100 may further include a blood pressure monitor 140. Furthermore, the blood pressure monitor 140 may be disposed within either the first band portion 104 or the second band portion 106 of the smartwatch assembly 100. The blood pressure monitor 140 may measure the systolic and diastolic blood pressures. The blood pressure monitor 140 may utilize a piezo-resistive pressure sensor. For example, the blood pressure monitor 140 may include a composite film that deforms and returns to its initial state when loading and unloading with pressure. Deforming and returning the composite structure to its initial state may decrease and increase resistance of the composite structure, and the resistance change is dependent on the pressure applied to the piezo-resistive pressure sensor. Accordingly, the piezo-resistive pressure sensor may detect a pulse in a wrist of the user and a blood pressure in the wrist of the user.

In one or more embodiments, the blood pressure monitor 140 may be disposed within band securing loop 141 (i.e., a free loop) of the first band portion 104 of the smartwatch assembly 100. For example, the blood pressure monitor 140 may be disposed within a portion of the band securing loop 141 oriented to remain in contact with the user's skin. Disposing the blood pressure monitor 140 within the band securing loop 141 of the first band portion 104 may assist in keeping the blood pressure monitor 140 against the skin of the user. In other embodiments, the blood pressure monitor 140 may be disposed within other portions of the band portions 104, 106 of the smartwatch assembly 100 or within the watch body 102 of the smartwatch assembly 100.

As noted above, the blood pressure monitor 140 may be operably coupled to the controller 136. Additionally, the controller 136 may cause data related to a measured blood pressure to be displayed on the display 120. For instance, the controller 136 may cause a numerical representation of a measured blood pressure to be displayed on the display 120. Moreover, in some embodiments, the controller 136 may cause the blood pressure monitor 140 to measure a blood pressure of the user. For instance, the controller 136 may, in response to a detection of one or more of the above described trigger events by the PPG sensor 118, cause the blood pressure monitor 140 to measure a blood pressure of the user. Moreover, a blood pressure measurement may be initiated by a user via one or more of the control mechanisms 122. Furthermore, the controller 136 may cause alerts to be displayed on the display 120 related to blood pressures measured by the blood pressure monitor 140. For instance, the controller 136 may cause alerts related to relatively high or low blood pressures to be displayed on the display 120.

FIG. 5 shows a schematic representation of a graphical user interface (“GUI”) shown by the display 120 of the smartwatch assembly 100. Referring to FIGS. 1A, 1B, 2, and 5 together, in some embodiments the display 120 may include an LCD screen. The GUI may include a main display area 144, a secondary display area 146, an icon area 148, and at least one RGB LED module 150. The main display area 144 of the GUI may show content (e.g., a time, a heart function, numerical representation of a tracked activity (e.g., steps taken)) related to a current selected mode of the smartwatch assembly 100 (described below). The secondary display area 146 of the GUI may display a date and/or additional content (e.g., an alarm time, durations of an activity session, data related to a tracked activity, etc.) related to a current selected mode of the smartwatch assembly 100. The icon area 148 may display a plurality of icons representing different current functions of the smartwatch assembly 100. For example, the icon area 148 may display an icon representing each of the following: battery charge level, current time, a heart function, a tracked activity of the user, and an alarm. Furthermore, an icon of the plurality of icons correlating to a currently displayed function and/or mode of the smartwatch assembly 100 may be highlighted (e.g., illuminated).

As noted above, the smartwatch assembly 100 may include a plurality of control mechanisms 122, and a user may utilize the plurality of control mechanisms 122 to operate functions of the smartwatch assembly 100. Table 1 below illustrates example functions that may be carried out by the smartwatch assembly 100 depending on whether the first button control mechanism 122A or the second button control mechanism 122B is pressed once for a short time (e.g., less than two seconds), referred to as a “press,” pressed twice successively, each for a short time, referred to as a “double press,” or pressed once for a long period of time (e.g., two seconds or more), referred to as a “long press.”

TABLE 1

| | | | 2 second | 5 second | 10 second |
|---|---|---|---|---|---|
| Mechanism | Press | Double Press | press | press | press |
| 122A | Switch | Switch Main | Switch | No Function | No Function |
| | Secondary | Display Area | Mode | | |
| | Display Area | | | | |
| 122B | Start/Stop | No Function | Turn on | No Function | Bluetooth Pair |
| | Tracking an | | Backlight | | |
| | Activity | | | | |
| 122A + | If menu HR | No Function | No Function | Start/Stop | No Function |
| 122B | or Activity, | | | Sleep | |
| | Switch | | | Tracking | |
| | between | | | | |
| | HR/QRS | | | | |

Of course, the functions set forth in Table 1 above and any other functions of the smartwatch 100 as described herein could be assigned to any of the control mechanisms 122A, 122B, and any other method or methods of initiating the functions using the control mechanisms 122A, 122B could be employed as well.

The smartwatch assembly 100 may have three operational modes. The first mode may include a time mode. In the first mode, the main display area 144 may show the time or the amount of steps taken. Furthermore, the second display area 146 may show the date, a time of an alarm, and the amount of steps taken. The second mode may include a sports mode. As noted above, a user may start tracking an activity or a sports session by pressing the second control mechanism 122B. In the second mode, the main display area 144 may show steps taken during the session and/or the current heart rate of the user. With the secondary display area 146, the user has the option of seeing (or the secondary display area 146 displaying) the duration of the session, the average and max heart rate, the VO₂ max, burned calories, steps taken during the sessions, a distance traveled in the session, and a target heart rate zone. The second mode may be paused by pressing the second control mechanism 122B again and resumed by pressing the second control mechanism 122B yet again. The second mode may be terminated by double pressing the second control mechanism 122B. The third mode may include a heart rate mode. In the third mode, a user may initiate a measurement via the PPG sensor 118, ECG sensor 116, or the blood pressure monitor 140 by pressing the second control mechanism 122B. In the third mode, the main display area 144 may indicate either a PPG measurement, an ECG measurement (e.g., parameters of the ECG measurement), or a measured blood pressure (e.g., systolic and diastolic pressures). The secondary display area 146 may show one or more of the following: a duration of the measurement and an average and max heart rate during the measurement.

The smartwatch assembly 100 may also have two operational states. The first mode is a normal state in which the watch functions normally as described herein. In this mode, the smartwatch assembly 100 tracks the activity of the user, the sleep of the user, it has a built in alarm, a built in time for different time zones, and a heartbeat related data measuring. The second state is a shipping mode in which the smartwatch assembly 100 “sleeps” and does nothing so as to conserve battery life. The smartwatch assembly 100 may be placed in shipping mode by pressing and holding the first button control mechanism 122A and the second button control mechanism 122B simultaneously for at least 10 seconds. The user may exit the shipping mode by holding the first button control mechanism 122A and the second button control mechanism 122B simultaneously for 10 seconds. The BLUETOOTH® functionality may also be activated upon exiting the shipping mode.

The smartwatch assembly 100 also includes a digital clock. The digital time may be equal to the time of an associated (e.g., “paired”) smartphone or other device. The digital watch of the smartwatch assembly 100 may be used for the activity tracking, inactivity tracking, alarm(s), etc. The digital time of the smartwatch assembly 100 may be automatically synced with the smartphone or other device whenever it is associated or otherwise paired with the smartphone or other device such that the digital time of the smartwatch assembly 100 is up to date, in case, for instance, the user changes time zones.

The smartwatch assembly 100 may track the activity of a user by counting the user's steps. The smartwatch assembly 100 may be capable of distinguishing the difference between running and walking by comparing the number of steps taken within a specified period of time. The user's weight, height, and gender may be considered in the algorithm for counting steps if the associated SW app allows the user to set a user profile including such information.

As a non-limiting example, the stride length formula may be defined as S=H×0.414, where S is the stride length in centimeters and H is the height of the person in centimeters. If the user's height H is not defined in the user's specific data, the default H may be set to 0.73 cm for a male and 0.67 for a female. This formula may be used to provide a more detailed activity and inactivity tracking dataset. Whenever there is no height H specified, the formula cannot be applied, in which case the standard male or female standard may be used. If the gender also is not provided, the standard value of 0.73 cm is used, for example.

The user's calorie expenditure through activity may be calculated within the smartwatch assembly 100 as long as the weight, height and gender of the user is provided within the user's profile in the SW app.

As previously mentioned, the smartwatch assembly 100 may be used to track the inactivity of the user. The user may wear the smartwatch assembly 100 while sleeping to track inactivity and, hence, quality of sleep. Sleep tracking can be turned on by either holding the both the first and second control mechanisms 122A, 122B for a long press (e.g., two seconds) as indicated in Table 1 above, or by turning on the sleep alarm using an associated SW app.

The smartwatch assembly 100 may include an alarm feature. For example, the smartwatch assembly 100 may include, for example, seven usual wake up/normal alarms and seven sleep alarms for the user to track his or her sleep. If a normal alarm goes off, inactivity tracking may also be disabled if it was previously enabled. The alarm(s) may be configured through the associated SW app. The alarm(s) may be configured differently for each day of the week if desired.

As noted above in regard to FIGS. 3A and 3B, the smartwatch assembly 100 may be used to provide indications to the user corresponding to notifications from an associated smartphone or other device, if connected to the smartwatch assembly 100. For example, the smartwatch assembly 100 may notify the user (e.g., notify the user via the vibrator or display 120) upon receiving incoming notifications from the associated smartphone or other device, and also notify the user upon receiving an incoming call on the associated smartphone or other device. As a non-limiting example, the smartwatch assembly 100 may notify the user of incoming calls, SMS messages, email messages, chat instant messages (e.g., WeChat instant messages, WhatsApp instant messages, etc.), social media notifications (e.g., FACEBOOK® notifications, TWITTER® notifications, etc.), and calendar events.

The smartwatch assembly 100 may also be found through the SW app by actuating a “Find My Watch” function in the SW app. If the smartwatch assembly 100 receives the BLUETOOTH® signal corresponding to the Find My Watch function, the smartwatch assembly 100 may, for example, vibrate for three seconds. Whenever the Find My Watch function is triggered from the smartwatch assembly 100, the smartphone or other associated device may vibrate and provide an audible sound signal.

The remaining battery life of the smartwatch assembly 100 may also be calculated and indicated to the user on the display 120. For example, if a battery level of the smartwatch assembly 100 is lower than 20%, the outline of the battery icon (described above) will appear empty and will illuminate. Moreover, when the battery is charging, the battery icon may include a charge symbol within the battery icon and will illuminate.

Any other features or functionalities described in relation to the smartwatch assembly 100 may also be employed in the smartwatch assembly 100, and the features or functionalities described in relation to the smartwatch assembly 100 may also be employed in the smartwatch assembly 100.

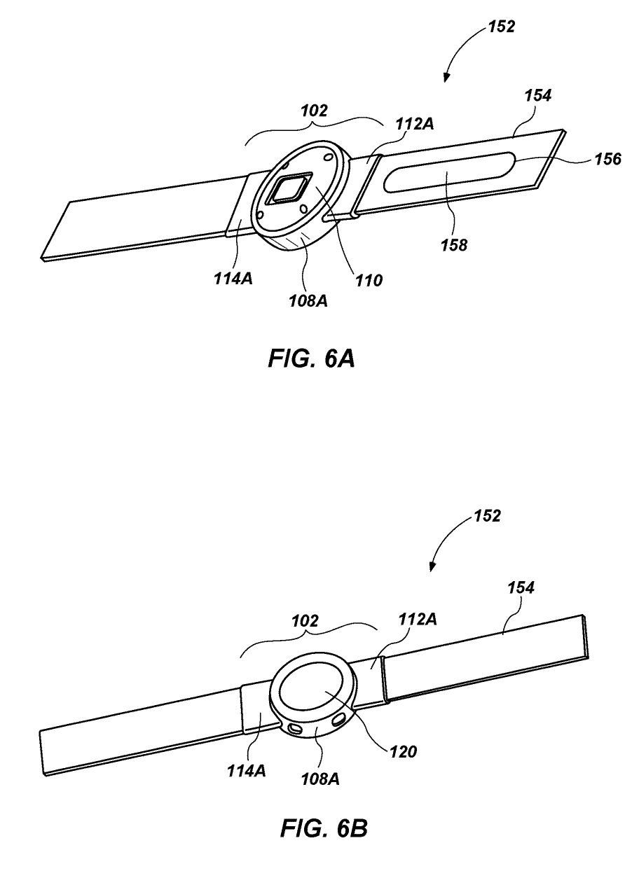

FIGS. 6A and 6B are perspective views of a smartwatch assembly 152 according to additional embodiments of the present disclosure. Similar to the smartwatch assembly 100 described above in regard to FIGS. 1A-5, the smartwatch assembly 152 may include a watch body 102. Furthermore, the watch body 102 may include a generally annular-outer frame portion 108A (e.g., receptacle portion), an insert portion 110, a first lug 112A, and a second lug 114A. Additionally, the insert portion 110 may be removably insertable into the outer frame portion 108A. However, the smartwatch assembly 100 may include a heart rate strap 154. The heart rate strap 154 may be sized and shaped to extend around and/or attached to a chest of a user. For instance, when a user wants and/or needs an accurate ECG measurement, the user may remove the insert portion 110 from the annular-outer frame portion 108 (FIG. 1A) of the smartwatch assembly 100 (FIG. 1A) and place the insert portion 110 within the annular-outer frame portion 108A of the smartwatch assembly 152. Moreover, the user may secure the heart rate strap 154 to the chest of the user with the watch body 102 being placed against the chest of the user and take an ECG measurement utilizing the smartwatch assembly 152 in any of the manners described above in regard to FIGS. 1A-5.

In some embodiments, the heart rate strap 154 may include at least one electrode 156 of the ECG sensor 116 of the smartwatch assembly 152. For example, the heart rate strap 154 may include one or more portions of conductive rubber 158 that comprise the at least one electrode 156 of the ECG sensor 116. Furthermore, the at least one electrode 156 (i.e., the conductive rubber 158) may be electrically connected to the controller 136 (FIG. 2) via one or more of the plurality of contacts 123 (FIGS. 3A and 3B). Having the at least one electrode 156 of the ECG sensor 116 being located within the heart rate strap 154 and being more proximate to a heart of the user (in comparison to being on the user's wrist) may expand locations where electrical activity is detected by the ECG sensor 116 and may enable a more accurate ECG measurement by the ECG sensor 116.

In view of the foregoing, and referring to FIGS. 1A-6 together, the smartwatch assemblies 100, 152 of the present disclosure may be advantageous over conventional smartwatch assemblies. For example, upon being notified by the smartwatch assembly 100 of a detected heart irregularity, the user may remove the insert portion 110 from the outer frame portion 108 of the smartwatch assembly 100 and may insert the insert portion 110 into the outer frame portion 108A of the smartwatch assembly 152 with the heart rate strap 154 to acquire a more accurate ECG measurement. Based on the more accurate ECG measurement, a user may decide whether or not to seek medical attention. Accordingly, the smartwatch assemblies 100, 152 of the present disclosure may provide a useful and potentially lifesaving monitor to users who may be prone to heart irregularities and/or heart attacks.

Referring again to FIGS. 1A-2, and in particular reference to the controller 136, computer-readable media can be any available media that can be accessed by the controller 136. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) include RAM, ROM, EEPROM, Flash memory, phase-change memory (“PCM”), other types of memory, other optical disc storage, magnetic disc storage or other magnetic storage devices, or any other medium that can be used to store desired program code means in the form of computer-executable instructions or data structures and which can be accessed by controller 136.

Further, upon reaching various computer system components, program code means in the form of computer-executable instructions or data structures can be transferred automatically from transmission media to non-transitory computer-readable storage media (devices) (or vice versa). For example, a computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special-purpose computer (e.g., the controller 136), or special-purpose processing device to perform a certain function or group of functions. In some embodiments, computer-executable instructions are executed on a general-purpose computer to turn the general-purpose computer into a special-purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Additional non-limiting example embodiments of the present disclosure are set forth below.

### Embodiment 1

A smartwatch assembly, comprising: an outer frame portion; an insert portion removably insertable into the outer frame portion, the insert portion comprising: a casing; a controller disposed within the casing; an electrocardiogram sensor operably coupled to the controller, the electrocardiogram sensor having at least two electrodes configured to be placed in contact with a user's skin; a photoplethysmography sensor operably coupled to the controller and oriented to face the user's skin; and a display operably coupled to the controller and configured to show data related to measurements taken by the electrocardiogram sensor and the photoplethysmography sensor, wherein the photoplethysmography sensor is configured to detect trigger events in a heart function of the user, and wherein, in response to the detection of a trigger event, the electrocardiogram sensor is configured to initiate an electrocardiogram measurement of the user.

### Embodiment 2

The smartwatch assembly of embodiment 1, further comprising a wrist band attached to the outer frame portion of the smartwatch assembly and comprising conductive rubber.

### Embodiment 3

The smartwatch assembly of embodiment 2, wherein the wrist band further comprises a blood pressure monitor.

### Embodiment 4

The smartwatch assembly of embodiment 1, wherein the display comprises an LCD screen.

### Embodiment 5

The smartwatch assembly of embodiment 1, wherein the at least two electrodes of the electrocardiogram sensor are exposed through a back cover of the insert portion.

### Embodiment 6

The smartwatch assembly of embodiment 1, wherein at least one electrode of the at least two electrodes is exposed on a lateral side of the smartwatch assembly through the outer frame portion of the smartwatch assembly and is accessible via an opposite hand of the user.

### Embodiment 7

The smartwatch assembly of embodiment 1, wherein the smartwatch assembly is configured to alert the user via the display when a detected trigger event includes an irregularity in heart functions.

### Embodiment 8

The smartwatch assembly of embodiment 1, wherein the smartwatch assembly is configured to show, via the display, a first color when an electrocardiogram measurement of the user is normal, a second color when an electrocardiogram measurement of the user indicates the possibility of a heart irregularity, and a third color when an electrocardiogram measurement of the user indicates a heart irregularity.

### Embodiment 9

A smartwatch assembly, comprising: an outer frame portion; an insert portion removably insertable into the outer frame portion, the insert portion comprising: a casing; a controller disposed within the casing; an electrocardiogram sensor operably coupled to the controller, the electrocardiogram sensor having at least two electrodes configured to be placed in contact with a user's skin; a photoplethysmography sensor operably coupled to the controller and oriented to face the user's skin; and a display operably coupled to the controller and configured to show data related to measurements taken by the electrocardiogram sensor and the photoplethysmography sensor, wherein the photoplethysmography sensor is configured to detect trigger events in a heart function of the user, wherein, in response to the detection of a trigger event, the smartwatch assembly is configured to alert the user of the detected trigger event via the display, and wherein, in response to a user interaction, the electrocardiogram sensor is configured to initiate an electrocardiogram measurement.

### Embodiment 10

The smartwatch assembly of embodiment 9, further comprising a strap attached to the outer frame portion and sized and shaped to extend at least partially around a user's chest.

### Embodiment 11

The smartwatch assembly of embodiment 10, wherein the strap comprises a conductive rubber portion.

### Embodiment 12

The smartwatch assembly of embodiment 11, wherein one of the at least two electrodes of the electrocardiogram sensor includes the conductive rubber portion.

### Embodiment 13

The smartwatch assembly of embodiment 12, wherein one of the at least two electrodes of the electrocardiogram sensor is exposed through a back cover of the insert portion.

### Embodiment 14

The smartwatch assembly of embodiment 9, further comprising a wrist band attached to the outer frame portion, the wrist band comprising a blood pressure monitor.

### Embodiment 15

The smartwatch assembly of embodiment 1, wherein at least one electrode of the at least two electrodes is exposed through the outer frame portion of the smartwatch assembly and is accessible via an opposite hand of the user.

### Embodiment 16

A smartwatch assembly system, comprising: at least one processor; and at least one non-transitory computer-readable storage medium storing instructions thereon that, when executed by the at least one processor, cause the system to: monitor a user's heart function via a photoplethysmography sensor; detect a trigger event related to the user's heart function via the photoplethysmography sensor; in response to detecting the trigger event, initiate an electrocardiogram measurement of the user via an electrocardiogram sensor; and show data related to the electrocardiogram measurement via a display.

### Embodiment 17

The smartwatch assembly system of embodiment 16, wherein the trigger event comprises an irregularity in heart function.

### Embodiment 18

The smartwatch assembly system of embodiment 16, further comprising instructions that, when executed by the at least one processor, cause the system to perform a blood pressure measurement of the user via a blood pressure monitor.

### Embodiment 19

The smartwatch assembly system of embodiment 16, wherein the system initiates an electrocardiogram measurement utilizing at least one electrode that is exposed on a lateral side of a smartwatch assembly through an outer frame portion of the smartwatch assembly.

### Embodiment 20

The smartwatch assembly system of embodiment 16, further comprising instructions that, when executed by the at least one processor, cause the system to show data related to the electrocardiogram measurement utilizing at least three different colors.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternative useful combinations of the elements described, will become apparent to those skilled in the art from the description. Such modifications and embodiments also fall within the scope of the appended claims and equivalents.

What is claimed is:

1. A smartwatch assembly, comprising:
an outer frame portion;
an insert portion removably insertable into the outer frame portion, the insert portion comprising:
a casing;
a controller disposed within the casing;
an electrocardiogram sensor operably coupled to the controller, the electrocardiogram sensor having at least two electrodes configured to be placed in contact with a user's skin;
a photoplethysmography sensor operably coupled to the controller and oriented to face the user's skin; and
a display operably coupled to the controller and configured to show data related to measurements taken by the electrocardiogram sensor and the photoplethysmography sensor,
wherein the photoplethysmography sensor is configured to detect trigger events in a heart function of the user, and
wherein, in response to the detection of a trigger event, the electrocardiogram sensor is configured to initiate an electrocardiogram measurement of the user.

2. The smartwatch assembly of claim 1, further comprising a wrist band attached to the outer frame portion of the smartwatch assembly.

3. The smartwatch assembly of claim 2, wherein the wrist band further comprises a blood pressure monitor.

4. The smartwatch assembly of claim 1, wherein the display comprises an LCD screen.

5. The smartwatch assembly of claim 1, wherein the at least two electrodes of the electrocardiogram sensor are exposed through a back cover of the insert portion.

6. The smartwatch assembly of claim 1, wherein at least one electrode of the at least two electrodes is exposed on a lateral side of the smartwatch assembly through the outer frame portion of the smartwatch assembly and is accessible via an opposite hand of the user.

7. The smartwatch assembly of claim 1, wherein the smartwatch assembly is configured to alert the user via the display when a detected trigger event includes an irregularity in heart functions.

8. The smartwatch assembly of claim 1, wherein the smartwatch assembly is configured to show, via the display, a first color when an electrocardiogram measurement of the user is normal, a second color when an electrocardiogram measurement of the user indicates the possibility of a heart irregularity, and a third color when an electrocardiogram measurement of the user indicates a heart irregularity.

9. A smartwatch assembly, comprising:
an outer frame portion;
an insert portion removably insertable into the outer frame portion, the insert portion comprising:
a casing;
a controller disposed within the casing;
an electrocardiogram sensor operably coupled to the controller, the electrocardiogram sensor having at least two electrodes configured to be placed in contact with a user's skin;
a photoplethysmography sensor operably coupled to the controller and oriented to face the user's skin; and
a display operably coupled to the controller and configured to show data related to measurements taken by the electrocardiogram sensor and the photoplethysmography sensor,
wherein the photoplethysmography sensor is configured to detect trigger events in a heart function of the user,
wherein, in response to the detection of a trigger event, the smartwatch assembly is configured to alert the user of the detected trigger event via the display, and
wherein, in response to a user interaction, the electrocardiogram sensor is configured to initiate an electrocardiogram measurement.

10. The smartwatch assembly of claim 9, further comprising a strap attached to the outer frame portion and sized and shaped to extend at least partially around a user's chest.

11. The smartwatch assembly of claim 10, wherein the strap comprises a conductive rubber portion.

12. The smartwatch assembly of claim 11, wherein one of the at least two electrodes of the electrocardiogram sensor includes the conductive rubber portion.

13. The smartwatch assembly of claim 12, wherein one of the at least two electrodes of the electrocardiogram sensor is exposed through a back cover of the insert portion.

14. The smartwatch assembly of claim 9, further comprising a wrist band attached to the outer frame portion, the wrist band comprising a blood pressure monitor.

15. The smartwatch assembly of claim 1, wherein at least one electrode of the at least two electrodes is exposed through the outer frame portion of the smartwatch assembly and is accessible via an opposite hand of the user.

16. A smartwatch assembly system, comprising:
at least one processor; and
at least one non-transitory computer-readable storage medium storing instructions thereon that, when executed by the at least one processor, cause the system to:
monitor a user's heart function via a photoplethysmography sensor;
detect a trigger event related to the user's heart function via the photoplethysmography sensor;
in response to detecting the trigger event, initiate an electrocardiogram measurement of the user via an electrocardiogram sensor; and
show data related to the electrocardiogram measurement via a display.

17. The smartwatch assembly system of claim 16, wherein the trigger event comprises an irregularity in heart function.

18. The smartwatch assembly system of claim 16, further comprising instructions that, when executed by the at least one processor, cause the system to perform a blood pressure measurement of the user via a blood pressure monitor.

19. The smartwatch assembly system of claim 16, wherein the system initiates an electrocardiogram measurement utilizing at least one electrode that is exposed on a lateral side of a smartwatch assembly through an outer frame portion of the smartwatch assembly.

20. The smartwatch assembly system of claim 16, further comprising instructions that, when executed by the at least one processor, cause the system to show data related to the electrocardiogram measurement utilizing at least three different colors.
